(19) United States
(12) Patent Application Publication
(10) Pub. No.: US 2023/0398005 A1
(43) Pub. Date: Dec. 14, 2023

(54) METHOD FOR CONTROLLING A PROSTHETIC FOOT

(71) Applicant: Otto Bock Healthcare Products GmbH, Vienna (AT)

(72) Inventors: Michael Ernst, Hardegsen (DE); Martin Seyr, Vienna (AT); Alexander Pappe, Vienna (AT); Dirk Seifert, Vienna (AT); Andreas Bohland, Vienna (AT); Thomas Hofmann, Vienna (AT)

(21) Appl. No.: 18/246,783

(22) PCT Filed: Sep. 22, 2021
(86) PCT No.: PCT/EP2021/076059

§ 371 Date: Mar. 27, 2023

(30) Foreign Application Priority Data
Sep. 28, 2020 (DE) 10 2020 125 256.0

(51) Int. Cl. A61F 2/76; A61F 2/66

(52) U.S. Cl. CPC A61F 2/76; A61F 2/66; A61F 2002/7625; A61F 2002/6614

(57) ABSTRACT

The invention relates to a method for controlling a prosthetic foot (10) which has an upper part (11) with a fastening element (13) for a proximal prosthetic component (20) and a foot part (12) mounted thereon so as to be pivotable about a pivot axis (15), comprising a resistance device (40) used to counteract a pivoting movement of the foot part (12) relative to the upper part (11) by way of an adjustable resistance (Am), and comprising a control device (70) which is coupled to the resistance device (40) and to at least one sensor (60) and by means of which the resistance to pivoting is set on the basis of sensor data, wherein ground contact of the foot part (12) with the ground is determined by way of at least one sensor (60), wherein the relative spatial position of the foot part (12) and/or of the upper part (11) is determined using an inertial measurement unit (30) and wherein the resistance (Am) to pivoting is altered on the basis of the presence or lack of ground contact and the determined relative spatial position and/or the determined route of the relative spatial position.

The invention relates to a method for controlling a prosthetic foot which has an upper part having a fastening element for a proximal prosthesis component and a foot part mounted thereon so as to be pivotable about a pivot axis, having a resistance device with which an adjustable resistance is presented against a pivoting movement of the foot part relative to the upper part, and having a control device which is coupled with the resistance device and with at least one sensor and by means of which the resistance against pivoting is set as a function of sensor data.

Prosthetic feet are part of a prosthetic treatment and are mounted either directly on a prosthetic socket or on a lower leg tube. Besides rigid prosthetic feet having a cushioned heel region, so-called SACH feet, there are complex prosthetic feet having spring arrangements and damper arrangements inside the prosthetic foot. Adjustment devices may be provided inside a prosthetic foot in order to modify properties that relate to treading and rolling.

Besides prosthetic feet arranged rigidly on a fastening section or a fastening device, there are prosthetic feet which are mounted in an articulated fashion on an upper part and have a prosthetic foot joint in order to allow pivoting about, in general, a pivot axis. In order to influence the treading and rolling behavior, resistance devices are provided which can be adjusted on the basis of sensor data. The resistance devices are for example fluid dampers having valves for influencing a flow resistance, resistance devices based on magnetorheological devices, in which the viscosity of a medium is modified by means of changes in a magnetic field, inter alia braking devices or drives which provide a resistance against a pivoting movement by corresponding interconnection. Starting from an initial status, the pivoting takes place in the direction of the floor with so-called plantar flexion, and in the opposite direction in the scope of dorsal flexion in which the toe of the foot is moved in the direction of the tibia. The adjustment of the respective resistances is carried out based on the evaluation of sensor data. The evaluation is carried out in a control device which is coupled with the sensors. The control device instigates suitable measures in order to adapt the resistance as desired, for example with valves being adjusted, magnetic fields being set up or motors being shifted into generator operation.

An example of a prosthetic foot and a control is disclosed in WO 2014/079588 A1. In this case, in the event of a knee flexion, a plantar flexion of a foot part is brought about by means of a force transmission device from an upper leg part. The force transmission may be carried out hydraulically, a valve block being switched as a function of a knee angle and a plantar flexion angle. Attitude sensors or force or moment sensors are provided in order to ascertain the attitude in space, forces or moments and use them for controlling the valves.

It is an object of the present invention to provide reliable and safe control of the resistance behavior of the prosthetic foot, particularly in situation transitions, for example at the start of walking after a standing phase.

According to the invention, this object is achieved by a method having the features of the main claim. Advantageous configurations and developments of the invention are disclosed in the dependent claims, in the description and in the figures.

According to the method for controlling a prosthetic foot, which has an upper part having a fastening element for a proximal prosthesis component and a foot part mounted thereon so as to be pivotable about a pivot axis, having a resistance device with which an adjustable resistance is presented against a pivoting movement of the foot part relative to the upper part, and having a control device which is coupled with the resistance device and with at least one sensor and by means of which the resistance against pivoting is set as a function of sensor data, ground contact of the foot part with the ground is determined by means of at least one sensor, the spatial attitude of the foot part and/or of the upper part is furthermore determined by means of an inertial measurement unit, and the resistance against pivoting is modified as a function of the presence or absence of ground contact and the spatial attitude that has been determined and/or the profile that has been determined of the spatial attitude. Because of the combined evaluation both of the spatial attitude and of the presence of ground contact, it is possible to allow improved adjustment of the respective resistances in the plantar flexion direction and dorsal flexion direction based on the sensor data and the situation of the prosthesis user which is deduced therefrom. In this case, both initially a modification may be calculated merely on the basis of ground contact data and a modification may be calculated on the basis of the spatial attitude data, and a superposition may take place, the safe resistance value in each case being set.

The presence or absence of ground contact may be determined by means of the recording of a ground reaction force, the ground reaction force being determined by means of a force sensor on the foot part, on the upper part and/or on a proximal prosthesis component. The ground reaction force may for example be determined by means of a contact switch, a pressure measurement film and/or a strain gauge. The arrangement of a ground reaction force sensor, in order to determine the state of whether or not there is ground contact, is a simple and reliable possibility for detecting the movement state respectively existing. In principle, it may be assumed that when a threshold value of an axial load on the prosthetic foot is fallen below, there is no longer a standing phase or at least the standing phase is about to be ended, that is to say the body weight has been shifted onto the contralateral side. If there is still a sufficiently large axial force proportion, ground contact may be assumed so that a corresponding resistance control against plantar flexion and/or dorsal flexion may be carried out. Beyond a threshold value being fallen below, it may be assumed that the standing phase has been initiated or is just being initiated, so that different resistances may optionally be set in the respective movement direction. The corresponding force sensors or moment sensors may be arranged directly on the foot part, on the upper part proximally with respect to the pivot axis, or on a proximal prosthesis component, which is connected to the upper part and therefore also to the lower part. A plurality of sensors for determining the ground contact by means of a ground reaction force or a corresponding moment may also be arranged on the prosthetic foot or on the proximal prosthesis component. Alternatively or in addition, the presence or absence of ground contact may be determined by means of at least one acceleration sensor on the foot part, on the upper part and/or on the proximal prosthesis component. While in general no movement of the prosthetic foot or at least parts of the prosthetic foot relative to the ground are carried out in the standing phase, in the swing phase the prosthetic foot is moved, in particular accelerated. The acceleration may in this case take place both in the vertical direction and in the horizontal direction, and it is likewise possible to determine the vertical acceleration or horizontal acceleration separately. When climbing stairs, for example, a pure vertical acceleration may take place by the vertical lifting of the prosthetic foot. After the lifting or after almost complete load relaxation, an almost horizontal acceleration of the prosthetic foot may occur because of a forward movement and may be determined by means of a corresponding acceleration sensor.

According to one development of the invention, a status of the foot part relative to the upper part and/or a pivoting movement of the foot part with respect to the upper part is determined, and the resistance against pivoting is modified as a function of the status and/or pivoting movement that has been determined. The ankle joint angle or the angle between the upper part and the lower part may, for example, be determined by means of an anklebone sensor or determination from spatial attitude data of the upper part and of the lower part. The pivoting movement of the foot part relative to the upper part may be determined by the same sensors. From the respective status as well as the variation of the status in the ankle joint, it is possible to draw conclusions about the presence or absence of ground contact, or a particular walking situation, so that these sensor values are likewise taken into account in the control of the resistance of the pivoting movement.

The magnitude of the ground reaction force may be measured, and the pivoting resistance may be increased, or pivoting may be blocked, when a threshold value of the ground reaction force is reached or exceeded. The ground reaction force may, as mentioned above, be determined directly by means of at least one contact switch, a pressure measurement film and/or at least one strain gauge, and/or by means of the deformation of a resilient element. The deformation of the resilient element may be recorded by means of a pressure sensor, a capacitive sensor, a length measurement, a volume measurement or a resistance device, so that the ground reaction force may be deduced therefrom. Furthermore, in addition or alternatively, by means of the recording of a rotational and/or translational relative position of two components flexibly connected to one another, or of the foot part and of another component, it is possible to determine whether there is a ground reaction force and optionally how great this ground reaction force is. Alternatively or in addition, the corresponding ground reaction force may be calculated by means of an ankle moment sensor from the moment and the assumed or determined force transmission point. There is likewise the possibility that the pivoting resistance is reduced for example when, by the ground reaction force falling below a threshold value, it is identified that a swing phase is initiated or a transition from walking on the flat to walking on a rising or ascending ground surface is taking place. It is likewise possible to identify that the prosthesis user is performing a movement which indicates a transition from walking on a flat surface or a ramp to a staircase.

The threshold value of the ground force may, for example, be set to between 5% and 50% of the body weight of the patient. If for example the threshold value of the ground reaction force falls below half the body weight of the patient, it may be inferred from this that a standing phase is being released and a swing phase is about to be initiated. If the prosthesis user is in the standing phase for a relatively long time, that is to say the user is standing for a relatively long period of time, it may be deduced from a reduction below a threshold value or from the speed of the reduction whether there is an intention to take a step in one direction, that is to say whether or not there is a movement intention. In combination with spatial attitude information of the upper part, of the foot part or of a proximal prosthesis component, the manner of the movement to be expected may then be deduced and a reduction, for example of the dorsal flexion resistance, may be carried out. If for example a threshold value substantially below 50% of the body weight of the patient is detected, or a change in the rotation rate or angular acceleration of the upper part or lower leg part is determined, or if the anklebone angle in the standing position is different than during walking, the flexion resistance is reduced so that it is possible to set off.

The angle between the foot part and the upper part or the proximal prosthesis component may be measured, the measured angle or the angle profile based on the measured angles, and the spatial attitude that has been determined of the upper part or of the proximal prosthesis component, being used in order to modify the resistance. The adjustment of the resistance is carried out in the standing phase, the respective situation being deduced from the anklebone angle and the inertial angle of the upper part, or of the proximal prosthesis component, and the adjustment of the resistance being carried out as a function of the values recorded. There is also the possibility in principle of only using the anklebone angle, that is to say the relative angle between the foot part and the upper part, or the proximal prosthesis component, and generating the corresponding adjustment of the resistance therefrom.

A dorsal movement may be enabled or prevented as a function of the measured angle between the upper part and the foot part and/or a spatial attitude angle. In this way, a dorsal flexion of the foot part is locked and increased safety is made possible for the prosthesis user because of the maximum control over the foot part.

The maximum dorsal flexion angle between the upper part and the foot part may be adjusted as a function of the ground surface inclination and/or the heel height. The adjustment of such a dorsal flexion stop may be carried out through a plurality of substeps up to the maximum dorsal flexion angle. The higher the heel is, that is to say the greater the heel height and therefore a variation of the zero-point attitude of the foot part relative to the upper part are, the more the dorsal flexion stop must be adjusted. The total possible dorsal flexion angle need not vary in this case. If a greater heel height is used, for example, the dorsal flexion stop in the plantar flexion direction is adjusted in order to generate an equal maximum dorsal flexion angle. If the heel height decreases, the dorsal flexion stop is advantageously adjusted in the dorsal flexion direction, the adjustment being carried out through a plurality of steps in individual substeps in order to allow slow adaptation to the changed external conditions, and therefore to make the adaptation as comfortable as possible for the prosthesis user.

A further dorsal flexion may be prevented when a limit value is reached for an inclination of the upper part or of the proximal prosthesis component, this also applying when a limit value is reached for the angle between the upper part and the lower part, the dorsal flexion respectively being prevented when one or other criterion is reached. The criterion respectively occurring earlier is therefore dominant, so that maximum safety is achieved for the prosthesis user.

During standing, a dorsal flexion may be locked when reaching a defined threshold value of the spatial orientation of the proximal prosthesis component.

During standing, a further dorsal flexion may be locked when reaching a defined threshold value of the spatial orientation of the upper part or of the proximal prosthesis component. Standing may for example be carried out by analyzing a profile of the force transmission point, that is to say the migration of a force transmission point between a front foot region and a heel region. If the force transmission point lies inside a region between two support points and if there is an axial load on both support points, a further dorsal flexion may be locked in order to achieve maximum stability. Likewise, in the event of a low speed of the force transmission point, it may be assumed that the prosthesis user is not in a walking situation but is in a standing situation, so that only a certain dorsal flexion is permitted during the standing function and standing situation.

The locking of the dorsal flexion may be released in the event of an increase in a ground reaction force to more than 50% of the patient's weight, and/or in the event of a change in the rotation rate of the upper part and/or in the event of the occurrence of an angular acceleration of the upper part. When at least one of these criteria occurs, it may be deduced that the standing situation is about to be terminated and that a step forward or backward is being instigated.

When walking on a downward sloping ground surface, the dorsal flexion stop is advantageously adjusted in the direction of an increased dorsal flexion angle, that is to say the stop is adjusted in the dorsal flexion direction in order to achieve a gait that is as natural as possible. Information about the ground surface inclination may, for example, be determined from the spatial attitude information of the foot part during full-surface contact of the sole of the shoe or the sole of the foot with the ground surface. If two force sensors are arranged in the region of the sole of the foot, for example, it is thereby possible to determine which phase of standing or standing phase a foot part is in. Likewise, by means of determining an ankle joint profile or an ankle moment profile, optionally in conjunction with IMU data of the upper part and/or of the lower part, it is possible to draw conclusions about the respective ground surface and the respective ground surface inclination. If a user of the prosthetic device is on a ramp leading upward, the dorsal flexion stop is adjusted and an increased dorsal flexion is made possible.

When walking on a downward sloping ground surface, the dorsal flexion stop may be increased by up to 500% compared with walking on the flat, and if there is a dorsal flexion stop normally starting from an initial value of for example 10°, when walking on a ramp this may be doubled or increased fivefold, that is to say a dorsal flexion value from 20° to 50° may be set.

In one development of the invention, the foot part is moved back into its initial status in a swing phase, which may be done by passive energy storage units, such as springs, which are loaded in the standing phase during the rolling movement, are optionally locked in the initial swing phase, and are released after swinging through, so that the prosthetic foot is correspondingly moved again when planting the foot, that is to say in the case of inertial ground contact. Alternatively or in addition to resetting by means of a passive energy storage unit, this may be carried out by means of an active drive in the form of a motor.

According to one development of the invention, the resistance of the resistance device is set to a minimum value in the event of a vertical orientation of the upper part, the resistance is preferably set to zero so that no additional resistance against pivoting in the plantar flexion direction and in the dorsal flexion direction is provided by the resistance device. Such control is advantageous both when walking on the flat and when walking on ramps, both upward and downward, so that in the event of a so-called zero crossing of the lower leg or of the upper part, free or almost free pivoting of the foot part is made possible. During a transition from plantar flexion to dorsal flexion with a set and/or modulated resistance, the moment crossover takes place during the zero crossing of the upper part or a vertical orientation of the upper part or of the lower leg.

Exemplary embodiments of the invention will be explained in more detail below with the aid of the figures, in which:

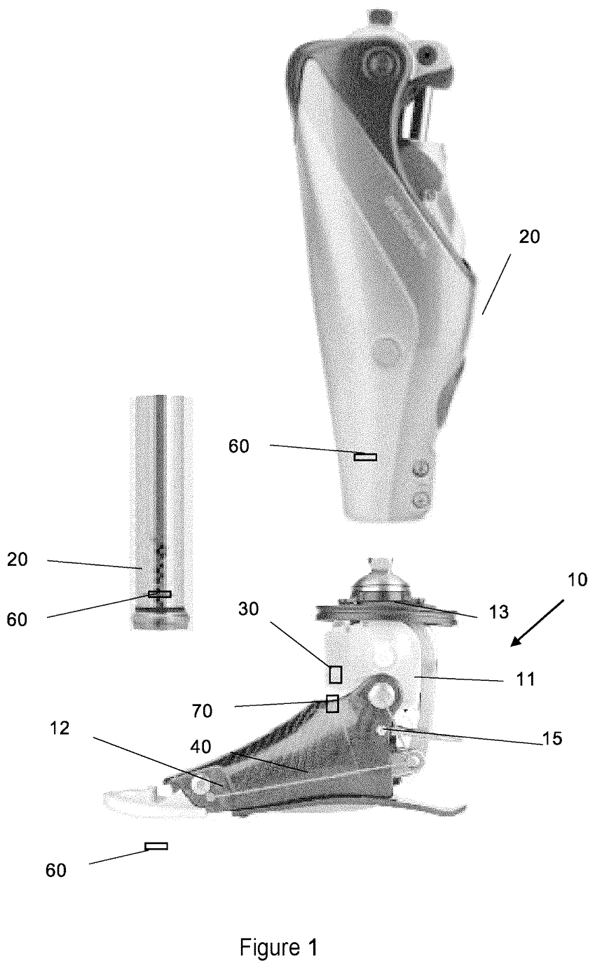

FIG. 1—shows a side view of a prosthetic foot with proximal components;

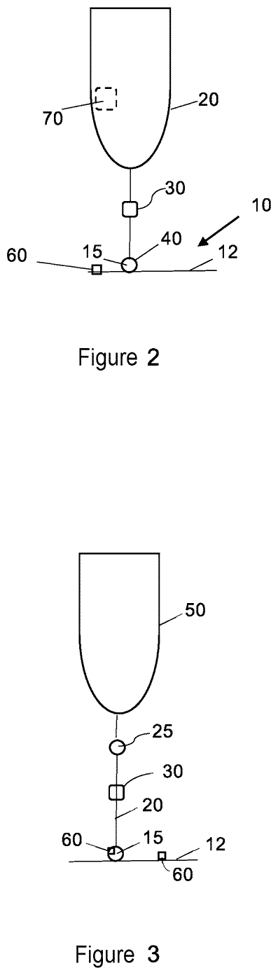

FIGS. 2 to 4—show schematic representations of prostheses with prosthetic feet;

FIG. 5—shows a control example;

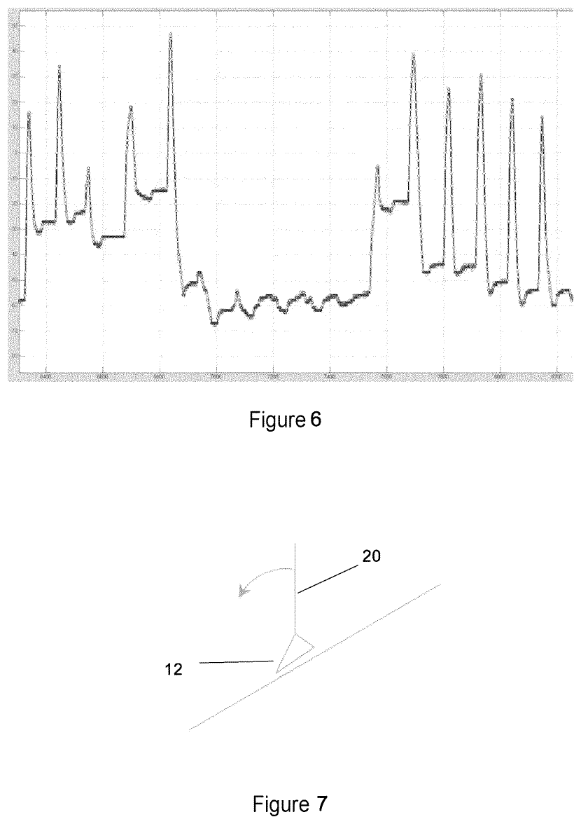

FIG. 6—shows an anklebone angle profile when walking on an incline;

FIG. 7—shows a schematic representation of a walking situation; and

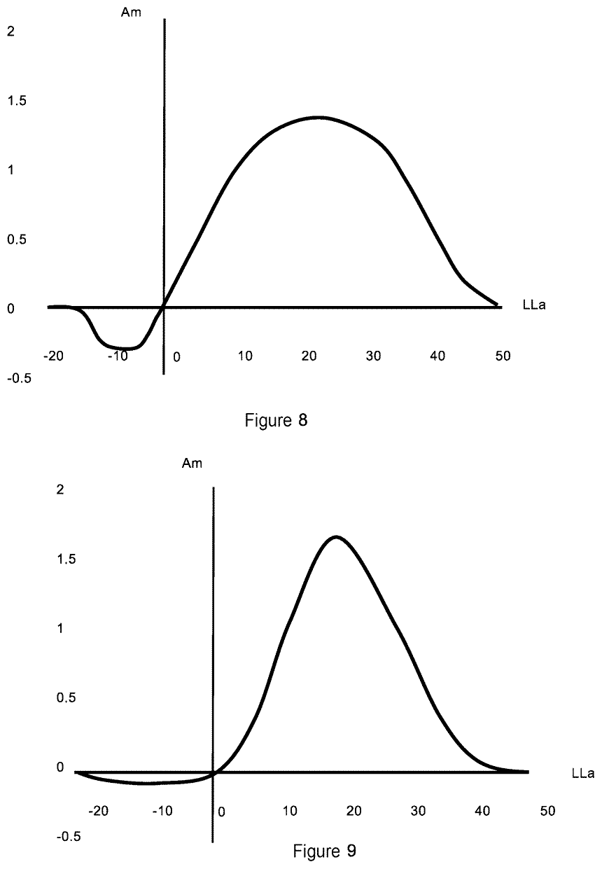

FIGS. 8 and 9—show resistance profiles as a function of the lower leg angle.

FIG. 1 represents a side view of a prosthetic foot 10 having an upper part 11 and a foot part 12, which are coupled with one another so as to be pivotable about a pivot axis 15. A fastening element 13 in the form of a pyramid adapter is arranged at the proximal end of the upper part 11. By means of the fastening element 13, it is possible to fasten proximal prosthesis components 20, for example a lower leg tube or a lower leg part having a prosthetic knee joint and an integrated damper, releasably onto the prosthetic foot 10. In the exemplary embodiment represented, the prosthetic foot 10 has precisely one pivot axis 15 in order to form an ankle joint. A sensor 60, which detects ground contact of the foot part 12, is arranged on the lower side of the foot part 12. The sensor 60 may be formed as an electrical contact element, as a pressure sensor, as a strain gauge or another configuration of a force sensor. In the exemplary embodiment represented, the ground contact sensor 60 is arranged in the forefoot region of the foot part 12. Alternatively or in addition, a ground contact sensor may also be arranged in the heel region or metatarsal region. Ground contact may also be determined by means of other sensors 60, for example by means of an axial force sensor which is arranged on the upper part 11 or on the fastening element 13. Likewise, a corresponding sensor device 60 may be arranged on the proximal prosthesis component 20, for example as a strain gauge or pressure sensor at a connecting point on the lower leg tube, on the lower leg part and/or on a knee part.

Arranged inside the prosthetic foot 10, there is furthermore a resistance device 40 with which it is possible to apply an adjustable resistance to plantar flexion or dorsal flexion. The resistance device 40 may be formed as a hydraulic damper, a damper on the basis of a magnetorheological liquid, as a mechanical brake or as a motor correspondingly operated in generator operation. In order to set the resistance device 40 based on sensor data and data of an inertial measurement unit 30 (IMU), the sensors or sensing devices 30, 60 are coupled with a control device 70, for example by means of a cable connection or wirelessly by means of a radio connection, in order to adjust or set the resistance against pivoting in one or the other direction about the pivot axis 15 as a function of the sensor data.

FIG. 2 shows a schematic representation of the prosthetic device with the prosthetic foot 10, the proximal prosthesis component 20 in the form of a lower leg socket, as well as the inertial angle sensor 30 or IMU fastened on the lower leg tube. The prosthetic foot 10 is mounted on the lower leg socket 20 so as to be pivotable by means of a joint about the axis 15. The upper part 11 and the fastening element 13 are not indicated for reasons of clarity. The IMU 30 is fixed either permanently or removably on the lower leg tube. A control device 70, which is coupled with the ground contact sensor 60, with the IMU 30 and with the resistance device 40, is furthermore arranged on the lower leg socket 20. The arrangement of the inertial angle sensor 30 or IMU may be carried out in a way that is integrated into or releasably fastened on a part, which is connected to the lower leg tube and the lower leg socket 20, of the prosthetic foot 10. It is likewise possible to arrange the sensor 60 not on the heel but on a metatarsal region, on the upper part or on the joint. Alternatively, a plurality of sensors 60 are arranged on the foot part 12.

FIG. 3 schematically represents a further prosthetic device, in which a second proximal prosthesis component 50 is arranged on the lower leg part 20. The second proximal prosthesis component 50 is, for example, an upper leg socket having a tube connecting to a prosthetic knee joint 25. In the exemplary embodiment represented, the IMU 30 is again arranged on the lower leg tube 20, although the inertial angle sensor 30 and optionally also the control device 40 may alternatively be integrated or releasably fastened on the knee joint 25 or on the upper leg socket 50 or on a connecting part between the upper leg socket 50 and the knee joint 25. Likewise, the IMU 30 may be arranged on an upper part of a prosthetic knee joint. In conjunction with an angle sensor, which records the angle between the lower leg part 20 and the proximal component 50, the spatial attitude of the lower leg part 20 may be detected from the spatial attitude of the second proximal component 50. Load, acceleration and/or angle sensors 60, which are arranged on the foot part 12 and on the ankle joint in the region of the pivot axis 15, are furthermore provided in the exemplary embodiment of FIG. 3. The load sensors 60 are for example axial force sensors, pressure sensors, for example in the form of contact switches, pressure measurement films and/or a strain gauge and/or torque sensors, in order to measure the respective load on the prosthetic device. The sensors 30, 60 are coupled with the control device 70 (not represented).

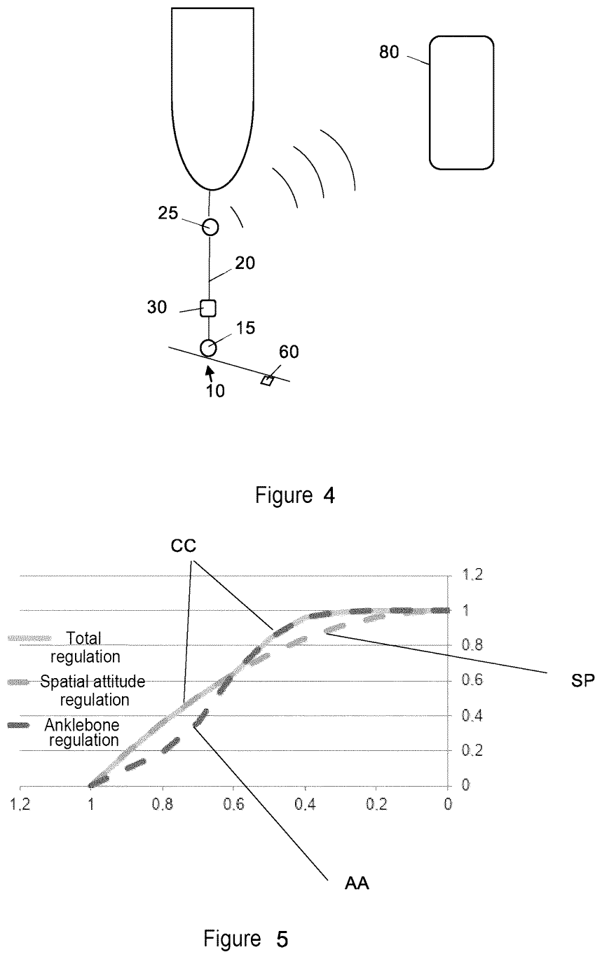

In FIG. 4, the control device 40 is formed disconnected and spatially separated from the inertial angle sensor 30 and is coupled with the prosthetic foot 10 by means of a wireless connection. The data transmission from the sensing devices to the control device 40 may take place by means of radio, WLAN, Bluetooth®, NFC or other transmission means.

With the sensors 60 and the IMU 30 the resistance against pivoting can be controlled precisely. The one sensor 30 determines the ground contact of the foot part 12 and therefore ensures that the prosthetic foot 10 is in the standing phase. At the same time, the spatial attitude of the foot part 12 and/or of the upper part 11 is determined by means of the IMU 30. If ground contact of the foot part 12 is detected, for example by a pressure sensor 60 fastened on the sole, the simultaneously determined spatial attitude of the foot part 12 and/or of the upper part 11 is recorded and used in order to control the resistance. This leads to superimposed control based on the spatial attitude of the foot part 12 and/or of the upper part 11 in conjunction with the detection of the standing phase, so that an increased reliability can be achieved during the setting of the respective resistance. This increased reliability is obtained particularly in situation transitions, that is to say when there are variations of the ground surface, for example when the ground surface inclination changes, or when the step rhythm varies, for example when changing from walking on the flat to climbing stairs or from walking on a ramp to walking on the flat or climbing stairs. In this case, the status of the upper part 11 relative to the foot part 12 may in particular also be taken into consideration as a further influencing quantity and sensor value. For this purpose, an ankle joint angle needs to be determined, for example by means of an angle sensor 60 directly on the pivot axis 15 or by means of the IMU 30 on the foot part 12 and on the upper part 11, or on a proximal prosthesis component 20 fastened thereto.

If for example, in the absence of ground contact of the foot part 12, which is determined via the ground reaction force by means of a force sensor on the foot part 12, on the upper part 11 and/or on the proximal prosthesis component 20, a spatial attitude change is detected by means of an IMU 30, whether it is a change in the spatial attitude of the proximal prosthesis component 20 or of the upper part 11 or of the foot part 12, this means for the control that a different resistance than in the standing phase is set in the resistance device 40 in a swing phase, which the prosthetic foot evidently is in. While in the event of a spatial attitude change, for example of the foot part 12, when there is a standing phase, it may be deduced that a rolling movement of the prosthetic foot has taken place and a terminal standing phase is assumed, in which the dorsal flexion is intended to be prevented and an increased dorsal flexion resistance must therefore be provided, for the same displacement of the foot part 11 in space during the swing phase a dorsal flexion may be desired in order to permit free swinging of the prosthetic foot, so that the dorsal flexion resistance is in this case reduced and an actuator is optionally activated in order to permit dorsal flexion of the foot part 12. The actuator may, for example, be formed as a passive energy storage unit which is unlocked in the swing phase so that resetting takes place at least into a neutral attitude. Alternatively, the actuator is an active drive or motor which moves the foot part back into its initial position.

The absence of ground contact may, alternatively or in addition, be determined by means of an acceleration sensor 60 on the foot part 12, on the upper part 11 and/or on the proximal prosthesis component 20. For example, if no acceleration of one of the two components takes place despite a load relaxation of the foot part 12 or of the upper part 11, a static state may be deduced, for example the existence of a standing phase. In the event of a pure vertical acceleration without a horizontal proportion, lifting of the prosthetic foot without a forward movement may be deduced, for example in order to step over an obstacle or in order to climb stairs. Based on the acceleration data of the acceleration sensor 60, it is possible to deduce either independently or in conjunction with a force sensor whether there is ground contact and which phase of the movement the respective prosthetic foot is in, if movement is identified.

Besides the absolute attitudes and the accelerations, it is likewise provided that the status of the foot part 12 relative to the upper part 11 and/or a pivoting movement of the foot part 12 relative to the upper part 11 is determined. The status may be determined by means of an angle sensor 60, and the pivoting movement is detected by means of the time derivative of the angle sensor data or by means of an acceleration sensor. The adjustment movement and the status of the foot part 12 relative to the upper part 11, and therefore also relative to the proximal prosthesis component 20 which is generally coupled rigidly on the upper part 11, also contributes to the reliability of the control.

FIG. 5 shows an example of a control based on the spatial attitude of the prosthetic foot, or of its components, and the angle between the foot part 12 and the upper part 11. Pure regulation by means of the anklebone angle, that is to say the status of the foot part 12 relative to the upper part 11, is represented in the lower curve with the broken line AA. Regulation only from the spatial attitude is represented in the broken line SP, and the total regulation is the solid line CC in the upper profile. Depending on the situation identified, a setpoint anklebone angle and a setpoint orientation of the upper part 11, or of the lower leg part 20, were respectively specified. A full match of the respective actual value with the setpoint angle gives the value 1, so that no adjustment is necessary, but depending on the degree of discrepancy the value changes and therefore so does the necessary adjustment of the resistance device. If the control were for example carried out only based on the spatial attitude curve SP, for a value of 0.4 a relatively small adjustment of the resistance value to approximately 0.8 would be necessary, while with unique control based on the anklebone angle and the curve AA an adjustment to 0.95 would be necessary. Conversely, for a value of 0.8 with unique control by means of the ankle joint angle and the curve AA, adjustment to 0.2 would be necessary, while with regulation by means of the spatial attitude based on the curve SP an adjustment to 0.35 would take place. For the respective control of the resistance device 40, the safer output value is set in each case, so that the upper curve CC which is represented by the solid line is obtained. Particularly during transitions from inclined ground surfaces to climbing stairs or in other transition situations, for example from walking on the flat to walking on a ramp, such a combination of the control of the resistance device 40 based on spatial attitude angles and relative angle values is particularly advantageous.

FIG. 6 represents the ankle joint angle, that is to say the angle between the foot part 12 and the upper part 11 over the course of a plurality of steps. Starting from an initial position, which is denoted by “0”, during normal walking on the flat a more or less uniform amplitude is executed above and below an initial status. Positive values indicate plantar flexion and negative values indicate dorsal flexion. The transition from walking on the flat to walking upward on a ramp takes place at the value of approximately 6900. The upper part needs to be displaced further in the direction of the foot part when walking uphill on a ramp, so that an increased dorsal flexion takes place. This is kept almost unchanged for walking on the ramp until, at 7500, a level place is reached once again and the resistance device can adapt again to walking on the flat.

The adjustment of the resistance device 40 may also be used to adjust the initial value for an adjustment around an initial attitude. By the shift of the zero point, around which the control of the resistance of the prosthetic device is carried out, adaptation may for example be carried out to different inclination angles in the ground surface or to different heel heights. Both for plantar flexion and for dorsal flexion, stops at which the resistance device 40 has and occupies a maximum value may be set in order to prevent further flexion. Thus, when reaching a limit value for an inclination of the upper part 11 or of the proximal prosthesis component 20 adjacent thereto, a further dorsal flexion can be prevented. The limit value for the inclination is in this case determined by means of the IMU 30 or from a combination of the spatial attitude values of the foot part and of an anklebone angle. Corresponding locking of the dorsal flexion may also be achieved when reaching a maximum dorsal flexion angle, the criterion respectively occurring earlier, that is to say the maximum spatial attitude or the maximum dorsal flexion angle, being the deciding factor for when locking is achieved.

FIG. 7 shows a schematic representation of the prosthetic foot having the foot part 12, which is mounted in an articulated fashion on the proximal prosthesis component 20. The foot part 12 is placed on a downwardly inclined plane. If the user of the prosthetic device is not in movement but is standing, dorsal flexion locking of the dorsal flexion may take place, for example with fixed vertical positioning, despite the usual initial status of the foot part 12 relative to the upper part 11 not being reached. If a variation of the heel height or a varying ground surface inclination is ascertained, for example because of significant discrepancies of the movement patterns and the angle profiles, as explained with the aid of FIG. 6, the adaptation and displacement of the zero status, around which dorsal flexion and plantar flexion take place, may be carried out stepwise so that an adaptation to a modified ground surface inclination takes place successively and the change amplitudes are not so great. If walking on an inclined ground surface is identified, the dorsal flexion may be adjusted accordingly. When walking on a downward ground surface, as represented in FIG. 7, the dorsal flexion stop may be adjusted in the direction of a reduced dorsal flexion angle, while with an opposite movement, that is to say uphill, the dorsal flexion stop may be adjusted in the direction of an increased dorsal flexion angle in order to accommodate the movement sequence according to FIG. 6.

The foot part 12 may in each case be moved back in the swing phase into an initial status, which forms the so-called zero attitude, around which dorsal flexion and plantar flexion are carried out with the corresponding resistance profile.

FIG. 8 represents a schematic representation of a resistance profile or of an ankle moment Am as a function of the lower leg angle LLa. The lower leg angle LLa corresponds substantially to the orientation of the upper part 11 of the prosthetic foot, since a proximal component such as a lower leg tube or the lower part of a prosthetic knee joint constitutes a rectilinear extension of the upper part. For a lower leg angle of 0°, the upper part and the lower leg part are in an initial status or vertical status, negative values corresponding to a plantar flexion of the foot part and positive values corresponding to a dorsal flexion.

The resistance or the ankle moment Am, which provides a resistance against displacement of the foot part relative to the upper part or the lower leg, is plotted as a function of the lower leg angle LLa. FIG. 8 represents the ascending of a ramp. After the heel strike and a relatively large plantar flexion, the foot part moves in the direction of an initial position, or a zero status, the ankle moment initially increasing and then decreasing when approaching the initial status, until it reaches a minimum value when reaching the initial status and a vertical orientation of the upper part. A minimum value may also be reached several times during a step cycle. In the event of an increasing dorsal flexion, the ankle moment and the resistance Am are increased until a maximum resistance value is reached for a lower leg angle of about 20 to 30° with respect to the vertical. Subsequently, the ankle moment decreases with increasing dorsal flexion.

FIG. 9 represents the resistance profile Am as a function of the lower leg angle when walking up a ramp. When walking up a ramp, no plantar flexion is generally to be expected, so that planting the foot on a rising ground surface starting from a normal status leads to dorsal flexion. Starting from this, an increase in the resistance Am or in the ankle moment takes place substantially more rapidly in comparison with walking down a ramp, so that the maximum value occurs in a range of between 10° and 20° dorsal flexions of the foot part. With an increasing dorsal flexion, the resistance Am is reduced until a maximum end stop is reached.

1-18. (canceled)

19. A method for controlling a prosthetic foot that comprises an upper part having a fastening element for a proximal prosthesis component and a foot part mounted thereon so as to be pivotable about a pivot axis, having a resistance device with which an adjustable resistance is presented against a pivoting movement of the foot part relative to the upper part, and having a control device which is coupled with the resistance device and with at least one sensor and by means of which the resistance against pivoting is set as a function of sensor data, the method comprising:
determining ground contact of the foot part with the ground by means of at least one sensor,
determining the spatial attitude of the foot part and/or of the upper part by means of an inertial measurement unit, and
modifying the resistance against pivoting as a function of at least one of the presence or absence of ground contact and the spatial attitude that has been determined or the profile that has been determined of the spatial attitude.

20. The method of claim 19, wherein the presence or absence of ground contact is determined by means of a ground reaction force, the ground reaction force being determined by means of at least one force sensor on at least one of the foot part, the upper part, or the proximal prosthesis component.

21. The method of claim 19, wherein the presence or absence of ground contact is determined by means of at least one acceleration sensor on at least one of the foot part, the upper part, or on the proximal prosthesis component.

22. The method of claim 19, further comprising determining at least one of a status of the foot part relative to the upper part or a pivoting movement of the foot part with respect to the upper part, and
modifying the resistance against pivoting as a function of at least one of the status or pivoting movement that has been determined.

23. The method of claim 19, wherein the ground reaction force is determined by one or more of at least one contact switch, a deformation of a resilient element, the recording of one or more of a rotational or translational relative position of two components connected flexibly to one another, a pressure measurement film or a strain gauge as the sensor.

24. The method of claim 19, wherein the magnitude of the ground reaction force is measured, and the pivoting resistance is reduced or increased, or pivoting is blocked, when a threshold value of the ground reaction force is reached or exceeded.

25. The method of claim 24, wherein the threshold value is set to between 5% and 50% of the body weight of the patient.

26. The method of claim 19, wherein the angle between the foot part and the upper part or the proximal prosthesis component is measured, and the measured angle or angle profile and the spatial attitude that has been determined of the upper part or of the proximal prosthesis component are used in order to modify the resistance.

27. The method of claim 26, wherein a dorsal movement is enabled or prevented as a function at least one of the measured angle between the upper part and the foot part or a spatial attitude angle.

28. The method of claim 27, wherein the maximum dorsal flexion angle between the upper part and the foot part is adjusted as a function of at least one of the ground surface inclination or heel height.

29. The method of claim 28, wherein the adjustment of a dorsal flexion stop is carried out through a plurality of substeps up to the maximum dorsal flexion angle.

30. The method of claim 26, wherein a further dorsal flexion is prevented when a limit value is reached for an inclination of the upper part or of the proximal prosthesis component, and a further dorsal flexion is prevented when a limit value is reached for the angle between the upper part and the foot part, the criterion occurring earlier being dominant.

31. The method of claim 19, wherein during standing, a dorsal flexion is locked when reaching a defined threshold value of the spatial orientation of the upper part or of the proximal prosthesis component.

32. The method of claim 31, wherein the locking of the dorsal flexion is released in the event of one or more of an increase in a ground reaction force to more than 50% of the patient's weight, a change in a rotation rate of the upper part or occurrence of an angular acceleration of the upper part.

33. The method of claim 19, wherein when walking on a downward sloping ground surface, a dorsal flexion stop is adjusted in the direction of an increased dorsal flexion angle.

34. The method of claim 33, wherein when walking on a downward sloping ground surface, the dorsal flexion stop is increased by from 100% to 500% compared with walking on the flat.

35. The method of claim 19, wherein the foot part is moved back into an initial status in a swing phase.

36. The method of claim 19, wherein the resistance of the resistance device is set to a minimum value in the event of a vertical orientation of the upper part.
